(19) United States
(12) Patent Application Publication
(10) Pub. No.: US 2022/0211260 A1
(43) Pub. Date: Jul. 7, 2022

(54) Ear Inspection Method

(71) Applicant: Ricknal Rhoden, Lithonia, GA (US)

(72) Inventor: Ricknal Rhoden, Lithonia, GA (US)

(21) Appl. No.: 17/140,029

(22) Filed: Jan. 1, 2021

(51) Int. Cl. A61B 1/227; A61B 1/00

(52) U.S. Cl. CPC A61B 1/227; A61B 1/00147

(57) ABSTRACT

A method for inspecting an ear of a user includes obtaining an inspection mirror having a handle, a first mirror and a second mirror, the first mirror being hingedly attached to the second mirror via a hinge, wherein the first mirror is moveable with respect to the second mirror to define a variable interior angle. The user grasps the handle of the inspection mirror, positions the inspection mirror behind the ear, adjusts the variable interior angle by pivoting the first mirror with respect to the second mirror, and looks into an additional mirror to view a reflection from the first mirror and the second mirror to see the interior of the ear of the user.

### BACKGROUND OF THE INVENTION

It can be difficult to view the interior of a person's ear, including the ear canal, in order to clean it or to inspect it for health reasons. Not only are the ears behind the eyes and directed outwardly, but the tragus further blocks a view into the ear canal from a position in front of the person. Consequently, the ear canal is best viewed from an angle generally behind the ear, looking in a direction extending from the back of the person's head toward the front of the person's head. Though a user may attempt to look into the ear canal with a simple hand-held mirror and then view a reflection in a wall mirror, this is a clumsy routine in which it is difficult to position and hold the mirror in the correct orientation.

### SUMMARY OF THE INVENTION

A method for inspecting an ear of a user includes obtaining an inspection mirror, in which the inspection mirror has a handle, a first mirror attached to the handle and a second mirror attached to the handle. The first mirror is hingedly attached to the second mirror via a hinge so that the first mirror is moveable with respect to the second mirror to define a variable interior angle.

In one version, the inspection method includes grasping, by the user, the handle of the inspection mirror, positioning the inspection mirror behind the ear of the user, adjusting the variable interior angle by pivoting the first mirror with respect to the second mirror, and looking into an additional mirror to view a reflection from the first mirror and the second mirror to view the interior of the ear of the user.

In some versions, the steps of grasping, positioning, adjusting, and looking are performed in sequential order, meaning the order stated above.

In other versions, the steps of grasping, positioning, adjusting, and looking are performed in any order, such that a user may, for example, position the mirror and then adjust it, or may look into the additional mirror and then adjust the first and second mirrors as necessary or desired.

In some versions, the handle of the inspection mirror further comprises a pivot joint, which may be a gimbal. In preferred versions, the pivot joint or gimbal is positioned adjacent the first mirror and the second mirror. Even more preferably, the handle includes a length from a distal end of the handle to a base of the hinge, the pivot joint being located at a position less than or equal to twenty percent of the distance from a base of the hinge to the distal end of the handle.

In some versions, the handle is an extendable handle, and may be telescopically extendable.

Most preferably, the additional mirror is a wall-mounted mirror.

In accordance with other versions of the invention, the method for inspecting an ear of a user includes obtaining or providing an inspection mirror, the inspection mirror having a handle, a first mirror being attached to the handle and a second mirror being attached to the handle, the first mirror further being hingedly attached to the second mirror via a hinge, wherein the first mirror is moveable with respect to the second mirror to define a variable interior angle.

The method further incudes grasping, by the user, the handle of the inspection mirror; positioning the inspection mirror behind the ear of the user; adjusting the variable interior angle by pivoting the first mirror with respect to the second mirror; and looking into an additional mirror to view a reflection from the first mirror and the second mirror to view the interior of the ear of the user.

Most preferably, the steps of grasping, positioning, adjusting, and looking are performed in any order, and the steps of adjusting and looking may be performed in an iterative fashion to adjust a viewing path and thereby improve the view of the interior of the ear of the user.

### BRIEF DESCRIPTION OF THE DRAWINGS

Preferred and alternative examples of the present invention are described in detail below with reference to the following drawings.

FIG. 1 is a front perspective view of a preferred inspection mirror, for use in performing a preferred ear inspection method.

FIG. 2 is a front perspective view of an alternated preferred inspection mirror, for use in performing a preferred ear inspection method.

FIG. 3 is a representative side view of a user shown holding a preferred inspection mirror adjacent a head of a user, in order to perform a preferred ear inspection method.

FIG. 4 is a top plan view of a user performing a preferred ear inspection method.

FIG. 5 is a flow diagram for a preferred ear inspection method.

### DETAILED DESCRIPTION OF THE PREFERRED EMBODIMENT

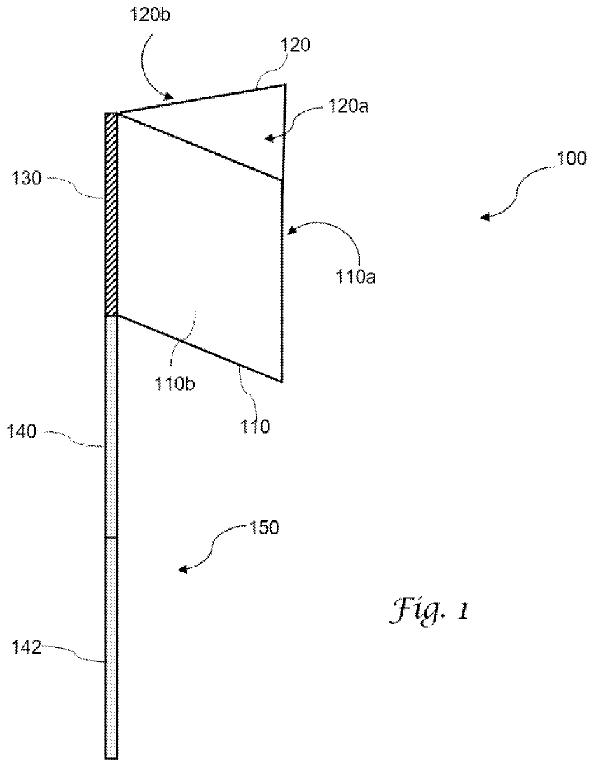

A preferred ear inspection mirror 100 is shown in a front perspective view in FIG. 1. In this version, the inspection mirror includes a handle 150, which may be an extendable or telescoping handle having a plurality of segments such as handle segments 140 and 142. In a preferred fashion, a first handle segment 140 may be telescopically received within, or may be extended from, a second handle segment 142. In some versions the handle may have any number of telescoping segments, or may be fixed in length, or may be extendable without handle segments which are telescopically extendable.

At a distal end, the inspection mirror 100 includes a pair of mirrors 110, 120. Each of the mirrors is hingedly attached to the distal end of the handle 150, via a hinge 130. The first mirror 110 includes a reflective inner surface 110a and an opaque outer surface 110b. Similarly, the second mirror 120 includes a reflective inner surface 120a and an opaque outer surface 120b. The first and second mirrors may pivotally move toward one another or way from one another, pivoting about the hinge which extends along a longitudinal axis defined by the hinge, which in a preferred version is also coaxial with the handle 150 as illustrated in the version of FIG. 1.

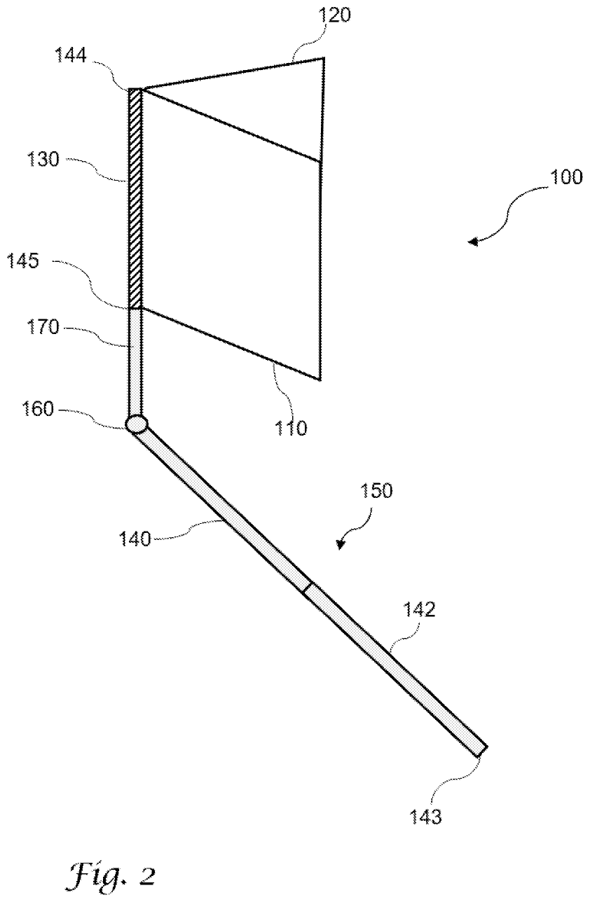

In some versions, such as illustrated in FIG. 2, the inspection mirror may include a pivot joint 160, which in some examples may be a gimbal, to provide an articulation point at some intermediate location of the handle. Preferably, the pivot joint or gimbal is relatively close to the mirrors 110, 120. In the version as illustrated, the handle comprises a third handle segment 170, which in this example is shorter than each of the first and second handle segments 140, 142. Thus, in the illustrated example the gimbal 160 is positioned between a proximal end 143 of the handle and a distal end 144 of the handle. More preferably, it is located more than 50 percent of the way from the proximal end 143 to the distal end. Even more preferably, the gimbal or pivot joint is located adjacent the base 145 of the hinge 130, such that the third handle segment which extends from the gimbal to the base 145 of the hinge 130 is less than 20 percent, and more preferably less than or equal to 10 percent, of the entire length of the handle which comprises the first, second, and third handle segments.

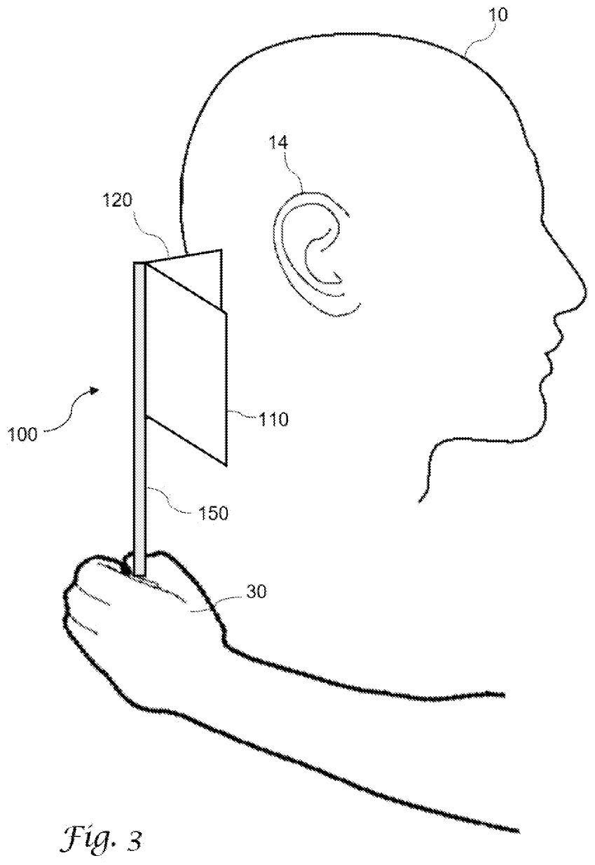

As seen in FIG. 3, a user preferably holds the inspection mirror 100 with the user's hand 30, extending the handle as desired, and bending the handle at the gimbal (not shown in FIG. 3 for simplicity) as desired. The inspection mirror is raised and positioned so that it is behind and adjacent the user's ear 14. The inspection mirror may be positioned on either side of the user's head 10, though only one side is shown.

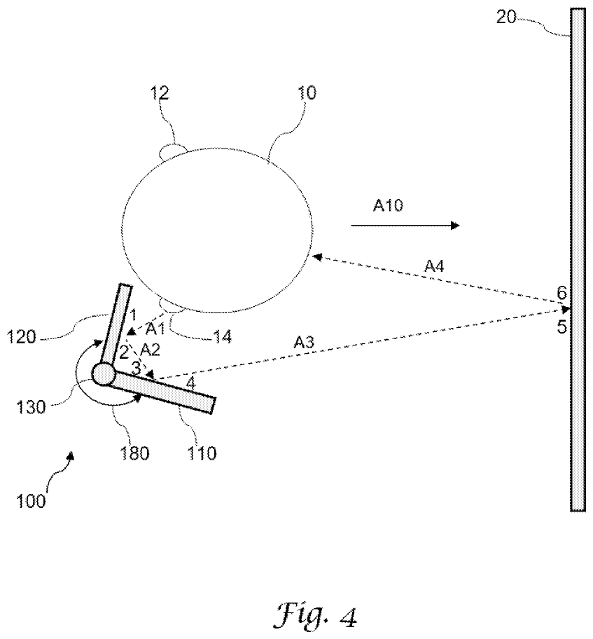

FIG. 4 is a top plan view of a preferred inspection method being performed by a user, intended to illustrate the same positioning as shown in FIG. 3 but without showing the user's hand or arm, for simplicity. The user's head 10 is shown with two ears 12, 14, and in this case a right ear 14 is shown undergoing inspection. The user may be standing or seated, and is shown looking forward in the direction of arrow A10 toward an additional mirror 20, which may be, for example, a typical bathroom mirror.

The inspection mirror 100 is illustrated in top plan view as well, showing the first mirror 120 and second mirror 120 pivotally attached at the hinge 130. The first and second mirrors may pivot toward or away from one another as desired, to create a desired interior angle between them. In FIG. 4, an exterior angle 180 is indicated for clarity of illustration, with the interior angle being explementary (that is, 360 degrees minus the exterior angle 180). The attachment of the first mirror to the second mirror at the hinge joint allows for a user-variable interior angle between them.

The first and second mirrors cooperate to allow the user 10 to see into the interior of the user's ear 14, as light traveling into the ear is reflected and travels outward along a first path A1 toward the second mirror 120. The image along the first path A1 is reflected by the second mirror to travel along a second path A2. As illustrated, the first path A1 and second path A2 are reflected from the second mirror 120 to define a first angle 1 and a second angle 2, which are equal to one another.

The image traveling along the second path A2 is reflected by the first mirror 110, and travels along a third path A3 toward the additional mirror. The image of the second path and the third path are reflected from the first mirror to define a third angle 3 and a fourth angle 4, which are equal to one another.

Finally, the inner ear image travels along the third path A3 toward the additional mirror, where it is reflected by the mirror 20 and travels toward the user's head 10 along a fourth path A4. The image of the third path A3 and the fourth path A4 are reflected from the additional mirror 20 to define a fifth angle 5 and a sixth angle 6 which are equal to one another.

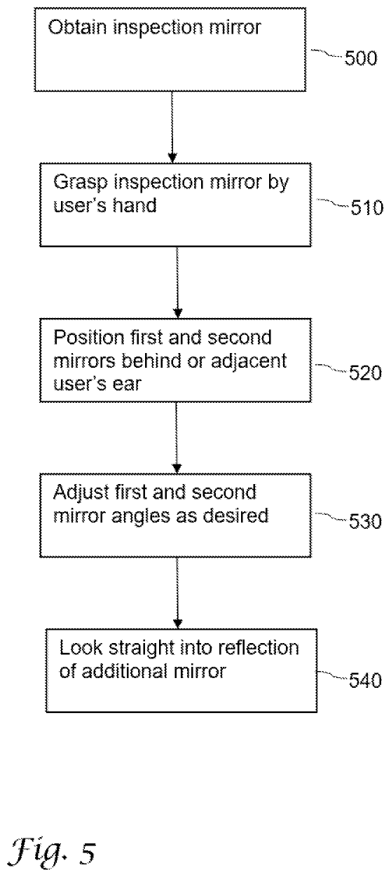

The process for using the inspection mirror to view a user's inner ear is further illustrated with reference to FIG. 5. In a first step 500, the user obtains an inspection mirror such as the preferred inspection mirror as described above. The user then, in a second step 510, grasp s the inspection mirror by the user's hand, preferably grasping the handle or a handle segment at or near the distal end. The handle is further extended, preferably telescopically, to a desired length.

In a third step 520, the user positions the inspection mirror, including the first and second mirrors, behind the head of the user and adjacent either a left or right ear. In a fourth step 530, the user adjusts the angle of the first mirror with respect to the second mirror as desired, in order to achieve image reflection and travel paths consistent with the description above. In a fifth step 540, the user is able to look straight into an additional mirror to see the reflection which contains the image into the interior of the ear under inspection.

It should be appreciated that the steps above can be performed in a different order, and can be iterative as part of an adjustment process. For example, the mirror angles can be adjusted prior to positioning the mirror behind the ear, and the handle can be extended afterwards as well. The mirror and handle positioning and adjustment can also be refined after looking into the additional mirror, to produce an improved angular orientation for inspection.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

I claim:

1. A method for inspecting an ear of a user, comprising:
obtaining or providing an inspection mirror, the inspection mirror having a handle, a first mirror being attached to the handle and a second mirror being attached to the handle, the first mirror further being hingedly attached to the second mirror via a hinge, wherein the first mirror is moveable with respect to the second mirror to define a variable interior angle;
grasping, by the user, the handle of the inspection mirror;
positioning the inspection mirror behind the ear of the user;
adjusting the variable interior angle by pivoting the first mirror with respect to the second mirror; and
looking into an additional mirror to view a reflection from the first mirror and the second mirror to view the interior of the ear of the user.

2. The method of claim 1, wherein the steps of grasping, positioning, adjusting, and looking are performed in sequential order.

3. The method of claim 1, wherein the steps of grasping, positioning, adjusting, and looking are performed in any order.

4. The method of claim 3, wherein the handle of the inspection mirror further comprises a pivot joint.

5. The method of claim 4, wherein the pivot joint is a gimbal.

6. The method of claim 4, wherein the pivot joint is positioned adjacent the first mirror and the second mirror.

7. The method of claim 4, wherein handle includes a length from a distal end of the handle to a base of the hinge, the pivot joint being located at a position less than or equal to twenty percent of the distance from a base of the hinge to the distal end of the handle.

8. The method of claim 3, wherein the handle is an extendable handle.

9. The method of claim 8, wherein the handle is telescopically extendable.

10. The method of claim 3, wherein the additional mirror is a wall-mounted mirror.

11. A method for inspecting an ear of a user, comprising:
obtaining or providing an inspection mirror, the inspection mirror having a handle, a first mirror being attached to the handle and a second mirror being attached to the handle, the first mirror further being hingedly attached to the second mirror via a hinge, wherein the first mirror is moveable with respect to the second mirror to define a variable interior angle;
grasping, by the user, the handle of the inspection mirror;
positioning the inspection mirror behind the ear of the user;
adjusting the variable interior angle by pivoting the first mirror with respect to the second mirror; and
looking into an additional mirror to view a reflection from the first mirror and the second mirror to view the interior of the ear of the user;
wherein the steps of grasping, positioning, adjusting, and looking are performed in any order, and further wherein the steps of adjusting and looking are performed in an iterative fashion to adjust a viewing path and thereby improve the view of the interior of the ear of the user.

12. The method of claim 11, wherein the handle is an extendable handle.

13. The method of claim 12, wherein the handle is telescopically extendable.

14. The method of claim 11, wherein the additional mirror is a wall-mounted mirror.
